(19) United States
(12) Patent Application Publication
(10) Pub. No.: US 2003/0105031 A1
(43) Pub. Date: Jun. 5, 2003

(54) Methods for the treatment of skin disorders

(76) Inventor: Richard A. Rosenbloom, Elkins Park, PA (US)

Correspondence Address: KNOBLE & YOSHIDA, EIGHT PENN CENTER, SUITE 1350, 1628 JOHN F KENNEDY BLVD, PHILADELPHIA, PA, 19103, US

(21) Appl. No.: 10/279,315

(22) Filed: Oct. 24, 2002

Related U.S. Application Data
(63) Continuation-in-part of application No. 10/132,642, filed on Apr. 25, 2002.
Application No. 10/132,642 is a continuation-in-part of application No. 10/045,790, filed on Jan. 14, 2002.
Application No. 10/045,790 is a continuation-in-part of application No. 09/993,003, filed on Nov. 6, 2001.

(51) Int. Cl. A61K 31/7048; A61K 31/353

(52) U.S. Cl. 514/27; 514/456

(57) ABSTRACT

Methods for the reduction, treatment or partial prevention of reactive and inflammatory dermatoses, including eczema and psoriasis, are provided. The methods comprise administering a composition that includes one or more flavonoids and is optionally formulated in a pharmaceutically acceptable carrier. Also provided are methods of facilitating the healing of wounds, and of cleansing, beautifying, and improving the cosmetic appearance of the skin. Further optional ingredients may be added to the composition used in the present invention, such as non-flavonoid antioxidants, and one or more compounds that regulate cell differentiation and/or cell proliferation. The composition may be administered as a topical composition.

### CROSS-REFERENCE TO RELATED APPLICATIONS

The present application is a continuation-in-part of U.S. application Ser. No. 10/132,642, filed on Apr. 25, 2002, which is a continuation-in-part of U.S. application Ser. No. 10/045,790, filed on Jan. 14, 2002, which is a continuation-in-part of U.S. application Ser. No. 09/993,003, filed on Nov. 6, 2001, each of which is incorporated herein by reference in its entirety.

### BACKGROUND OF THE INVENTION

1. Field of the Invention

The present invention relates to compositions and methods for treating skin disorders including reactive and inflammatory dermatoses such as eczema and psoriasis, to compositions and methods for treating wounds, and to compositions and methods for improving the cosmetic appearance of the skin.

2. Description of the Prior Art

Reactive and inflammatory dermatoses are non-contagious disorders of the skin whose causes, when known, are usually related to allergic or other immune reactions. These disorders may take the form of mild irritation; however, in more severe cases, reactive and inflammatory dermatoses may be extremely painful, disfiguring, and debilitating conditions.

Examples of reactive and inflammatory dermatoses include, without limitation, vesicular palmoplantar eczema, dyshidriotic eczema, lichen simplex chronicus, pruritic urticarial papules and plaques of pregnancy, lichen planus, granuloma annulare, allergic contact dermatitis, nummular dermatitis, seborrheic dermatitis, atopic dermatitis, and asteatotic eczema.

Eczema and psoriasis are two common examples of reactive and inflammatory dermatoses. These two skin conditions, together, affect hundreds of millions of people. Three percent of children under age five, up to one-fifth of school-age children, and about 8% of the adult population are afflicted with some type of eczema. Eczema includes a group of conditions also known collectively as dermatitis, and may be caused by exposure to allergens or irritants, by blood circulatory problems, or by a genetically-linked condition known as atopy. Atopy is characterized by an excessive immune reaction to allergens to which most people are not sensitive.

Eczema has a variety of symptoms. In its milder forms, the skin becomes dry, hot, and itchy. More severe symptoms include broken, raw, and weeping or bleeding skin that is particularly vulnerable to infection.

When eczema is caused by irritants or allergens, the condition may be alleviated by eliminating exposure to the irritant or allergen. There is no cure currently available for other forms of eczema. Treatments center on alleviating the symptoms, primarily itching and inflammation. Thus, the use of topical emollients is recommended, or cold wet compresses, and the avoidance of harsh soaps and woolen clothing. Topical steroids may also provide some relief. Systemic steroids are used for severe flare-ups. When the inflamed skin becomes infected, topical or systemic antibiotics are indicated.

Psoriasis is a chronic, genetic, non-contagious skin disorder that affects approximately one hundred million people worldwide. Any area of the skin may be affected, although psoriasis is most commonly found on the scalp, elbows, knees, hands, feet, and genitals. Psoriasis may be almost unnoticeable in its early stages, but plaque psoriasis, for example, usually begins with small red bumps on the skin that progress to bigger, scaly patches that may become itchy and uncomfortable. Patients often report an itching and or burning sensation as the disease progresses. As the scales accumulate, pink to deep red plaques with a white crust of silvery scales appear on the skin surface.

Other types of psoriasis are characterized by different signs and symptoms. For example, pustular psoriasis is characterized by pus-like blisters, erythrodermic psoriasis is characterized by intense redness and swelling of a large part of the skin surface, guttate psoriasis is characterized by small, drop-like lesions, and inverse psoriasis is characterized by smooth red lesions in the folds of the skin.

The cause of psoriasis is unknown, although it has recently been hypothesized that abnormal white blood cells trigger inflammation of the skin and concomitant rapid shedding of skin cells. The many palliative treatments available include moisturizing creams and lotions, topical steroids, localized steroid injections, coal tar, anthralin, salicylic acid, vitamin D and related compounds, vitamin A and other retinoids, methotrexate, cyclosporine, hydroxyurea, and controlled UV exposure. There is, however, no cure for psoriasis.

It is therefore apparent that a need remains for effective compositions and methods to treat eczema and psoriasis.

Accordingly, it is an objective of certain embodiments of the present invention to provide a topical composition that, when applied to the skin, will reduce, treat or at least partially prevent eczema and/or psoriasis.

It is a further objective of certain embodiments of the present invention to provide methods of reducing, treating or at least partially preventing eczema and/or psoriasis.

It is a further objective of certain embodiments of the present invention to provide methods of reducing, treating or at least partially preventing eczema and/or psoriasis by administering a topical composition.

It is a further objective of certain embodiments of the present invention to provide methods of facilitating the healing of wounds.

It is a further objective of certain embodiments of the present invention to provide methods of cleansing, beautifying, and improving the cosmetic appearance of the skin.

These and other objects of the present invention will be apparent from the summary and detailed descriptions of the invention, which follow.

### SUMMARY OF THE INVENTION

In a first aspect, the present invention relates to a method of reducing, treating or at least partially preventing eczema and psoriasis by administering a composition including a flavonoid and, optionally, a pharmaceutically acceptable carrier.

In another aspect, the composition is administered topically.

In another aspect, a method of facilitating the healing of wounds is provided. In this method, a composition including a flavonoid and, optionally, a pharmaceutically acceptable carrier is administered topically to a wounded patient.

In another aspect, a method of cleansing, beautifying, and improving the cosmetic appearance of the skin by topical administration of a composition including a flavonoid and, optionally, a pharmaceutically acceptable carrier is provided.

### DETAILED DESCRIPTION OF THE PREFERRED EMBODIMENTS

As used herein, a “pharmaceutically acceptable” component is one that is suitable for use with humans and/or animals without undue adverse side effects commensurate with a reasonable benefit/risk ratio.

Further, as used herein, the term “safe and effective amount” refers to the quantity of a component, which is sufficient to yield a desired therapeutic response without undue adverse side effects commensurate with a reasonable benefit/risk ratio when used in the manner of this invention.

“Adverse side effects” include, without limitation, toxicity, irritation, and allergic response. The specific “safe and effective amount” will, therefore, vary with such factors as the particular condition being treated, the physical condition of the patient, the duration of the treatment, the nature of concurrent therapy, if any, and the specific formulations employed.

As used herein, the term “daily dosage” refers to an amount of material administered to a patient during each day of the treatment. When the material is administered topically, daily dosages are assumed to apply to a treatment area measuring 100 cm², unless another size of treatment area is specified.

In certain embodiments of the invention, methods for the reduction, treatment, or at least partial prevention of eczema and/or psoriasis are provided. In the methods of the present invention, a composition comprising a therapeutically effective amount of a flavonoid is administered to a patient afflicted with eczema and/or psoriasis. The composition may optionally include a pharmaceutically acceptable carrier. The composition may also optionally include a non-flavonoid antioxidant. Preferably, the composition used in the present invention is administered topically.

It has now surprisingly been found that the methods of the present invention are effective to reduce, treat or at least partially prevent eczema and/or psoriasis. The compositions used in the methods of the present invention include at least one flavonoid. Flavonoids are small organic compounds having a phenyl benzopyrone structure. They are found in the leaves, fruits, seeds, stems, or flowers of all vascular plants. Citrus fruits are a prominent source of flavonoids, over 4000 of which have been identified as deriving from plant sources. On average, the daily Western diet contains about one gram of mixed flavonoids.

Without wishing to be held to any hypothesis, flavonoids have antioxidant properties and anti-inflammatory effects that are believed to aid in the treatment of reactive and inflammatory dermatoses. Also, flavonoids have the property of protecting certain cells and tissues, for example ischemic tissue and hypoxic tissue. Finally, flavonoids inhibit histamines and leukotrienes, chemicals that contribute to atopic reactions.

Examples of flavonoids include, without limitation, flavonones, flavanols, anthocyanidins, proanthocyanidins, procyanidolic oligomers, biflavans, rutinosides, hydroxyethylrutinosides, and leucoanthocyanins. The choice of specific flavonoids to be included in the composition may be determined by factors such as toxicity, bioavailability, solubility or dispersability, and the like. Also, flavonoids include compounds derived from known flavonoids, which compounds retain at least some of the desired activity of the flavonoid from which it is derived.

Examples of flavonoids suitable for use in the present invention include, without limitation, 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; pycnogenol, quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; and wogonin, and the pharmaceutically acceptable salts and solvates thereof.

Another preferred flavonoid is green tea polyphenol or green tea extract, which contains compounds such as (−)-epigallocatechin-3-gallate, (−)-epigallocatechin-3-gallate, (−)-epigallocatechin and/or (−)-epicatechin. Studies (see Elmets, C. A. et al, J. Am. Acad. Dermatol., 44 (3); 425-32, March, 2001) have shown that green tea polyphenol or extract is effective in inhibiting erythema.

The flavonoid used in the composition of the present invention may also include one or more curcuminoids. Examples of curcuminoids include, without limitation, curcumin (diferuloylmethane), desmethoxycurcumin (hydroxycinnamoyl feruloylmethane), and/or bis-desmethoxycurcumin (dihydroxydicinnamoyl methane) (see Drug Analysis by Chromatography and Microscopy, p. 169, Ann Arbor Science Inc., 1973). These compounds may be purchased from commercial sources or isolated from turmeric. Methods for isolating curcuminoids from turmeric are known, (see Janaki and Bose, An Improved Method for the Isolation of Curcumin From Turmeric, J. Indian Chem. Soc. 44:985 (1967)). Alternatively, curcuminoids for use in the present invention can be prepared by synthetic methods. Curcuminoids possess antioxidant properties and also have anti-inflammatory, anti-tumor and other valuable properties.

Preferred flavonoids will also impart additional beneficial effects in the composition of the present invention. For example, some of the flavonoids may act as radical scavengers to decrease the concentration of free radicals such as hydroxyl radicals.

More preferred flavonoids are quercetin, quercetrin, myricetin, kaempferol, rutin, green tea extract, and myrecetrin. These compounds may have some anti-inflammatory activity and/or may help stabilize cell membranes in combination with a relatively low toxicity, both of which activities may be beneficial in the treatment of eczema and/or psoriasis.

Preferably, the composition used in the present invention also includes a non-flavonoid antioxidant. The non-flavonoid antioxidant may be a single compound or material or a mixture of two or more compounds and/or materials. Compounds and materials which may be used as non-flavonoid antioxidants in the present invention are those that exhibit antioxidant activity when administered to a patient, without causing any severe adverse side affects when used in an amount effective to provide sufficient antioxidant activity. Preferably, the non-flavonoid antioxidant does not react with one or more of the ingredients of the composition resulting in a substantial loss of activity of one or more of the ingredients. Preferred non-flavonoid antioxidants are materials obtained from plants or other natural sources, or are derivatives of such materials.

The non-flavonoid antioxidant used in the composition is present in an amount effective to provide significant antioxidant activity when administered to a patient in the composition of the present invention.

Preferred non-flavonoid antioxidants are selected from ascorbic acid (vitamin C), its esters, for example, ascorbyl palmitate, and other compounds having vitamin C activity such as those generally called Ester-C™ that are disclosed in U.S. Pat. Nos. 4,822,816 and 5,070,085; vitamin A and its esters, for example, vitamin A palmitate; vitamin E and its esters, for example, vitamin E acetate; lipoic acid, preferably α-lipoic acid, and more preferably DL-α-lipoic acid; carotenoids such as β-carotene; chlorophyllin and its salts; coenzyme Q10; glutathione; L-dopa, cysteine, N-acetyl cysteine, cystine, pangamic acid (dimethyl glycine), taurine, tyrosine, carbohydrates such as beta-1,3-glucan, germanium, alpha-hydroxy acids including glycolic acid and lactic acid, phytic acid (inositol hexaphosphate), caffeic acid (3,4-dihydroxy-cinnamic acid), ellagic acid, ferulic acid, gallic acid, gamma-oryzanol, resveratrol (trans-3,5,4′-trihydroxystilbene), zingerone (4-(4-hydroxy-3-methoxyphenyl)-2-butanone), nordihydroguaiaretic acid, pyrroloquinoline quinone, allicin, dithiolthiones, glucosilinates, S-allyl-L-cysteine, tocotrienols, carnosol, and the pharmaceutically acceptable salts, solvates, and derivatives thereof which exhibit antioxidant activity.

Mixtures of two or more non-flavonoid antioxidants may also be employed in the composition used in the present invention. Preferred non-flavonoid antioxidant mixtures are ascorbyl palmitate with one or more of vitamin A, vitamin E acetate and α-lipoic acid, especially DL-α-lipoic acid. The antioxidants may also be used in the form of their pharmaceutically acceptable salts and this may be preferred in some cases to increase solubility or dispersability, to reduce adverse side effects, etc.

In another preferred embodiment, the antioxidants used in the composition of the present invention includes at least one of the compositions having vitamin C activity disclosed in U.S. Pat. Nos. 4,822,816 and 5,070, 085, which are also commonly called Ester-C™. The Ester-C™ described in these patents generally includes an effective amount of a compound having vitamin C activity.

As used herein, the term, “compound having vitamin C activity” means vitamin C (L-ascorbic acid) and any derivatives thereof exhibiting antiscorbutic activity. Vitamin C derivatives include, for example, oxidation products, such as dehydroascorbic acid and edible salts of ascorbic acid such as, e.g., calcium, sodium, magnesium, potassium and zinc ascorbates, esters of vitamin C with organic and inorganic acids, such as L-ascorbic acid 2-0-sulfate, L-ascorbic acid 2-0-phosphate, L-ascorbic acid 3-0-phosphate, L-ascorbic acid 6-hexadecanoate, L-ascorbic acid monostearate, L-ascorbic acid dipalmitate and the like.

Metabolites of ascorbic acid and its derivatives include the aldonic acids, aldono-lactones, aldono-lactides and edible salts of aldonic acids. Preferably, the compound having vitamin C activity includes one or more of these metabolites selected from L-threonic acid, L-xylonic acid and L-lyxonic acid. The presence of one or more of these metabolites in the compositions of the invention may provide an improvement in absorption and/or retention of vitamin C or other therapeutically active compounds.

Structurally similar derivatives of one or more of these compounds, which exhibit antioxidant activity when administered in the oral compositions of the present invention, may also be employed.

By “structurally similar derivatives” is meant derivatives that exhibit antioxidant activity and contain at least one significant, common structural element with the compound or material from which it is derived.

In a preferred embodiment, the non-flavonoid antioxidant used in the composition of the present invention may include one or more antioxidant enzymes. The antioxidant enzymes useful in the present invention are those capable of scavenging radicals, promoting radical scavengers or preventing radical formation. The preferred antioxidant enzymes useful in the present invention include superoxide dismutase, catalase, glutathione peroxidase, glutathione-S-transferase, and methionine reductase. Other antioxidant enzymes with activities similar to those mentioned above may also be used. In addition, one or more of the antioxidant enzymes may act in combination with one or more of the antioxidant compounds in the composition to, for example, scavenge free radicals and/or at least partially prevent cell damage in the skin.

The non-flavonoid antioxidants used in the composition of the present invention are preferably selected not only for their antioxidant activity, but also based on other beneficial effects that particular compounds may provide. For example, a racemic mixture of α-lipoic acid not only has a strong antioxidant activity but also has a recycling effect on vitamins C and E, and thus is a particularly preferred antioxidant for the present invention. In addition, α-lipoic acid can function in both lipid and non-lipid environments.

Vitamin C and its esters are not only antioxidants, but also exhibit a strong combinatorial effect with vitamin E and its esters when used together. In fact, vitamin E and its esters, and vitamin C and its esters can mutually reinforce one another by a mechanism in which one antioxidant (reducing agent) acts as a regenerator for the oxidized form of the other.

Vitamin A is a fat-soluble material and thus is preferred for use due to this additional beneficial property. Preferably, vitamin A may be used in its ester forms, such as vitamin A palmitate, because the ester forms of vitamin A may be less irritating to the stomach.

Carotenoids such as β-carotene may also be included in the composition of the present invention as preferred antioxidants. Several carotenoids have shown beneficial effects for the present application, such as enhancement of immune response. Carotenoids can also protect against photo-induced tissue damage. Some carotenoids, including β-carotene, quench highly reactive singlet oxygen under certain conditions and can block free radical-mediated reactions.

Preferably, the antioxidant used in the composition of the present invention also includes chlorophyllin and/or its salts. More preferably, chlorophyllin and its salts may be included in the composition of the present invention in amounts, which, when administered to a patient according to a method of the present invention, provide a daily dosage between about 20 milligrams and about 500 milligrams. Chlorophyllin and its salts may be an alfalfa extract or extracted from silkworm feces. Chlorophyllin and its salts may also be purchased from common commercial sources such as Aldrich Chemical Company of St. Louis, Mo.

Also preferably, the antioxidant used in the composition of the present invention includes a combination of effective amounts of vitamin A or its esters, vitamin C or its esters, vitamin E and α-lipoic acid to achieve the beneficial effect of recycling vitamin C or its esters and vitamin E by α-lipoic acid.

Some of the non-flavonoid antioxidants useful in the present invention are more active in a lipid environment whereas others are more active in a non-lipid environment. Accordingly, the composition of the present invention preferably includes a combination of at least two antioxidants, with one being selected for its higher activity in a lipid environment and a second one being selected for its higher activity in a non-lipid environment.

When the composition is formulated into a topical composition, the antioxidant enzyme used in the present invention is preferably skin-absorbable. However, depending on its solubility characteristics, the antioxidant enzyme may need to be formulated in a suitable dispersant such as corn oil in much the same manner as vitamin D₃, as described below. Alternatively, liposomes or the like may be sufficient to incorporate or stabilize an antioxidant enzyme in formulation.

A compound that regulates cell differentiation and/or cell proliferation may optionally be included in the composition of the present invention. The compound that regulates cell differentiation and/or cell proliferation is used in an amount effective to regulate cell differentiation and/or cell proliferation when orally administered to a patient in the oral composition of the present invention.

Suitable compounds that regulate cell differentiation and/or cell proliferation are those that do not induce significant, adverse side effects when administered to a patient in amounts that regulate cell differentiation and/or cell proliferation, and which do not react with one or more of the ingredients of the composition resulting in a substantial loss of activity of one or more of the ingredients. Preferred compounds for regulating cell differentiation and/or cell proliferation are those that occur naturally in the human body and/or materials obtained from plants or animals which may be administered to humans without significant, adverse side effects in the amounts used, or derivatives thereof.

More preferably, the compounds that regulate cell differentiation and/or cell proliferation used in the present invention inhibit or prevent cell differentiation or cell proliferation. Even more preferably, the compounds that regulate cell differentiation and/or cell proliferation used in the present invention accomplish at least one of the following: maintenance of cellular homeostasis and normal cell metabolism, regulation of cell differentiation, induce certain cancer cells to differentiate into normal cells, preferably by working in combination with vitamin A, maintenance of the epidermal permeability barrier, inhibition of cancer cell differentiation, and inhibition of cancer cell proliferation.

Methods for screening compounds that regulate cell differentiation and/or cell proliferation are well-known. For example, DiscoveRx Corporation at Fremont, Calif. markets a Hithunter™ tyrosine kinase assay to detect inhibitors of tyrosine kinase and tyrosine phosphatase which control or regulates cellular growth, proliferation and differentiation using β-galactosidase EFC activity. In this assay, inactive fragments of galactosidase, enzyme acceptor (EA) and enzyme donor (ED) complement to form active enzyme. Binding of an ED-conjugated peptide to an antibody inhibits complementation, while unlabeled peptide displaces the ED-conjugate. This results in increased β-galactosidase activity that is detected subsequently either chemiluminescence or long wavelength fluorescent substrates.

Hithunter™ tyrosine kinase assay has been developed to measure activity of the human insulin receptor, EGF receptor kinase domains and Src (EC 50=2.8 nM, 4.4 nM and 4.9 nM respectively). Hithunter™ tyrosine phosphatase activity was also measured using PTP 1B enzyme (EC 50=48 nM). Assay performance characteristics (Z′=0.5-0.7, CV=5-8%) and a simple two step addition protocol make it ideal for HTS (high throughput screening).

Another exemplary method for screening compounds that regulate cell differentiation and/or cell proliferation is available from the Commercial Ventures & Intellectual Property Office at University of Massachusetts, Worcester, Mass. The method can be used to screen for cancer drugs and other drugs that inhibit or promote cell growth, cell death or cell differentiation for diseases involving ERb action, including prostate, breast and ovarian cancer, neurological disorders, osteoporosis and cardiovascular disease. In the method, the effect of any compound on ER-beta regulated cell growth/cell death/cell cycle arrest is determined by adding the compound to culture cells expressing the receptor and measuring alteration in expression levels of ER-beta regulated genes.

Exemplary compounds that regulate cell differentiation and/or cell proliferation are vitamin D₃, vitamin D₃ analogs, compounds that may be converted or metabolized into vitamin D₃ in the human body, and metabolites thereof. Exemplary compounds that may be converted or metabolized into vitamin D₃ include common cholesterols illustrated below. The cholesterol illustrated below may be converted into provitamin D when a hydrogen is removed from the number 7 carbon, which then forms a double bond with the number 8 carbon, in the second, or ‘B’ ring of the cholesterol molecule. The cholesterol is oxidized, that is, an electron is removed with the hydrogen atom, so that the double bond is a consequence of 2 mutually shared electrons between carbons 7 and 8.

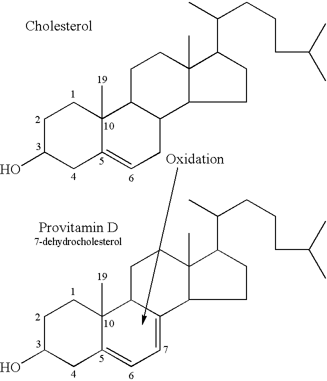

Provitamin D may be converted to vitamin D₃ by the action of ultraviolet light through human skin. In this reaction, the B ring of the sterol molecule is opened.

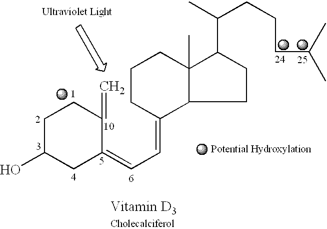

Cholecalciferol, which is vitamin D₃, may be further converted into another vitamin D intermediate, 25-hydroxcholecalciferol, in the liver by mitochondrial hydroxylase, in the presence of NADPH, and molecular oxygen.

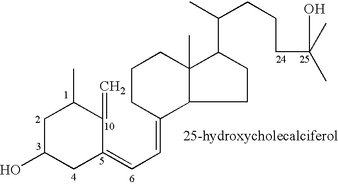

When more active vitamin D₃ is required, 25-hydroxycholecalciferol is transported to the kidney where a new hydrolase enzyme is synthesized. This enzyme introduces another hydroxyl group at position 1, and the bioactive form of vitamin D₃, calcitriol, is produced.

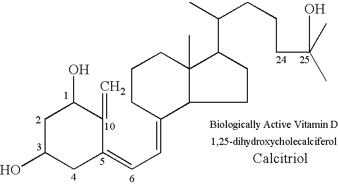

Examples of a suitable vitamin D₃ analogs include, without limitation, 1(S), 3(R)-dihydroxy-20(R)-(1ethoxy-5-ethyl-5-hydroxy-2-heptyn-1-yl)-9, 10-seco-pregna-5(Z), 7(E), 10 (19)-triene. Examples of suitable vitamin D₃ metabolites include, without limitation, 1, 25-dihydroxyvitamin D₃. Also, pharmaceutically acceptable salts of the compounds that regulate cell differentiation and/or cell proliferation may be employed. Preferably, the compound that regulates cell differentiation and/or cell proliferation comprises vitamin D₃.

It may be advantageous to use a dispersant in formulating the compound that regulates cell differentiation and/or cell proliferation in the composition of the present invention. Such compounds are often only slightly soluble in water. Suitable dispersants are known to persons skilled in the art. One suitable dispersant for the compound that regulates cell differentiation and/or cell proliferation is corn oil. Corn oil also has the advantage of being a natural product. The corn oil is used in an amount sufficient to disperse the compound that regulates cell differentiation and/or cell proliferation.

The composition for use in the present invention may provide one or more of the following beneficial effects to a patient when administered in an effective amount: antioxidant properties, free radical scavenging, transition metal chelation, nitric oxide stabilization, anti-inflammatory activity, relief of pain, burning, tingling, electrical sensations and/or hyperalgesia, increased microcirculation, nitric oxide stabilization, promotion of healing of skin ulcers and lesions, protein kinase C inhibition, decreased oxidative stress, anti-inflammation, protection against radiation damage, blockage of the formation of leukotrienes, stabilization of cell membranes, and regulation of cell differentiation, cell proliferation protection of mitochondrial membranes, reduction of cell damage, especially damage to DNA molecules, and may play a role in the repair and regeneration processes of damages cells.

In a method for treating or reducing eczema and psoriasis, an effective amount of the topical composition comprising a flavonoid is applied one to twelve times daily, as needed, to an area of skin afflicted with eczema and/or psoriasis. Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

The method of the present invention may provide one or more of the beneficial effects described above for the compositions used in the invention. In addition, the method of the present invention may provide one or more additional beneficial effects due to one or more of the ingredients contained in the pharmaceutically acceptable oral or topical carriers as described below.

The pharmaceutically acceptable carrier used in the present invention may be a carrier suitable for use as a carrier for topical compositions. The non-carrier ingredients are dissolved, dispersed and/or suspended in the topical composition. Examples of suitable topical carriers include, without limitation, creams, ointments, lotions, pastes, jellies, sprays, aerosols, bath oils, and other topical pharmaceutical carriers, which accomplish direct contact between the active ingredients of the topical composition of the present invention and the pore of the skin. Preferably, the pharmaceutically acceptable topical carrier may make up more than about 80%, and more preferably about 80-95% w/w of the total composition. In some cases, it may be necessary to dissolve one or more the active ingredients in an appropriate solvent such as ethanol or DMSO (dimethylsulfoxide), or the like, to facilitate the incorporation of one or more active ingredients into the topical composition or the pharmaceutically acceptable topical carrier. Preferably, an amount of topical carrier sufficient to provide a substantially homogeneous cream or ointment is used.

One preferred topical carrier useful in the present invention contains at least a hydrophilic ointment base, panthenol or a panthenol derivative and a dispersant if needed to disperse one or more insoluble or partially insoluble active ingredients in the carrier. Another preferred topical carrier of the present invention employs hydroxymethyl cellulose as the base and may contain ingredients contained in the carrier described below other than the hydrophilic ointment base.

Yet another preferred pharmaceutically acceptable topical carrier includes a solution of an acrylic copolymer in a non-aqueous solvent system, which mainly contains polyethylene glycol such as methoxy polyethylene glycol 550 (MPEG). A particular preferred MPEG is Sentry Carbowax MPEG 550 sold by the Dow Chemical Company of Midland, Mich., which is a food/pharmaceutical/cosmetic grade material. Polyethylene glycols are generally non-toxic, water-soluble polymers that are fully biodegradable. In the solution, the acrylic copolymer would preferably be present in a concentration range of 3-6% by weight. Preferably, the acrylic copolymer has a molecular weight of more than 20,000 Daltons. More preferably, the acrylic copolymer has a molecular weight of more than 100,000 Daltons. Acrylic polymers having higher molecular weights are less likely to be absorbed by the human body or skin. Other pharmaceutically acceptable carrier components, other than the hydrophilic ointment base may also be employed in this carrier material, if compatible with the acrylic copolymer.

Suitable hydrophilic ointment bases are known to persons skilled in the art. Examples of hydrophilic ointment bases suitable for use in the present invention include, without limitation, non-U.S.P. hydrophilic ointment bases such as those made by Fougera, Inc., of Melville, N.Y. Sufficient hydrophilic ointment base is employed to act as a topical carrier for the active or non-carrier ingredients of the topical composition.

Panthenol or panthenol derivatives are preferably included in the pharmaceutically acceptable carrier. The panthenols useful in the present invention include at least D-panthenol, DL-panthenol and mixtures thereof. This component of the topical carrier has skin moisturizing properties and acts as a quick, deep penetrating component of the topical carrier that helps deliver the non-carrier ingredients through the skin to the area to be treated and may also impart a healing effect to damaged tissue. The amount of panthenol or panthenol derivative to be employed is from about 0.25 to about 10 weight percent, more preferably from about 0.5 to about 5 weight percent and most preferably from about 1 to about 2 weight percent, based on the total weight of the topical composition.

The topical carrier of the present invention may also include additional ingredients such as other carriers, moisturizers, humectants, emollients, dispersants, radiation blocking compounds, particularly UV-blockers, as well as other suitable materials that do not have a significant adverse effect on the activity of the topical composition. Preferred additional ingredients for inclusion in the topical carrier are sodium acid phosphate moisturizer, witch hazel extract, glycerine humectant, other humectants such as Ajidew NL-50, available from Ajinomoto USA, Inc., of Paramus, N.J., apricot kernel oil emollient, and corn oil dispersant.

The topical composition used in the present invention may also be employed to facilitate wound healing. The compositions may also be used to cleanse, beautify, and improve the cosmetic appearance of the skin. In fact, one measure of a therapeutically effective amount of the various compositions of the present invention is by determining if that amount improves the healing of, or the cosmetic appearance of the skin. More preferably, the compositions of the present invention are employed to improve the healing of, or the cosmetic appearance of the skin in the area afflicted by reactive and inflammatory dermatoses such as eczema and/or psoriasis.

In a method for wound healing, an effective amount of the topical composition comprising a flavonoid is applied one to twelve times daily, as needed, to a wound. More preferably, an effective amount of the topical composition comprising a flavonoid is applied to an area of skin afflicted with reactive and inflammatory dermatoses such as eczema and/or psoriasis. Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

In a method for cleansing, beautifying, and improving the cosmetic appearance of the skin, an effective amount of the topical composition comprising a flavonoid is applied one to twelve times daily, as needed, to an area of skin. More preferably, an effective amount of the topical composition comprising a flavonoid is applied to an area of skin afflicted with reactive and inflammatory dermatoses such as eczema and/or psoriasis. Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

The topical composition of the present invention is preferably made by cold compounding. This is an important feature of the invention when one or more of the compounds employed in the topical composition is sensitive to heat or other types of energy. In these cases the activity of the topical composition may be detrimentally affected as a result of the formulation of the topical compositions in a manner that allows excess heat to be generated during compounding. It may be necessary to dissolve, disperse or suspend one or more of the ingredients prior to cold compounding in order to ensure substantially homogeneous distribution of the non-carrier or active ingredients in the topical composition.

A preferred pharmaceutically acceptable topical carrier of the invention can be made using the following ingredients: 25-35 parts of a 50% aqueous solution of sodium acid phosphate moisturizing agent, 5-10 parts of D- or DL-panthenol, 5-10 parts of glycerine, 1-3 parts of apricot kernel oil and 10-20 parts of witch hazel extract.

Optionally, one or more of the optionally ingredients of the topical composition such as glycerin, witch hazel extract, vitamins A and E and/or the ascorbyl palmitate can be reduced or eliminated from a particular topical composition, if desirable, or larger amounts of one type of component, i.e. an antioxidant, can be employed while reducing the amount of another component of the same type or having a similar activity.

When the composition of the present invention is formulated into a topical composition, preferably, the vitamins A and D₃ used in the composition of the present invention may be formulated in a single corn oil dispersion. Generally, every cubic centimeter (cc) of the corn oil dispersion of vitamins A and D₃ used in the present invention may contain about 500,000 to about 2,000,000 IU of vitamin A and about 50,000 to about 200,000 IU of vitamin D₃. Preferably, every cc of the corn oil dispersion of vitamins A and D₃ used in the present invention may contain about 1,000,000 IU of vitamin A and about 100,000 IU of vitamin D₃.

A preferred composition for use in the present invention, based on about 1 lb of hydrophilic ointment base, includes 1-20 grams of flavonoid and 100 mg to about 5 grams of a non-flavonoid antioxidant. More preferably, based on 1 lb of hydrophilic ointment base, this composition contains 2-10 grams of flavonoid and 500 mg to 2 grams of non-flavonoid antioxidant.

An even more preferred composition includes quercetin dihydrate (about 1 to about 20 g, more preferably about 2 to about 4 g), DL-α-lipoic acid (about 100 mg to about 10 g, more preferably about 500 mg to about 1 g), green tea (based on a content of 36-50% catechins and polyphenols, about 100 mg to about 10 g, more preferably about 500 mg to about 1 g), and rutin (about 0.1 to about 10 g, more preferably about 1 g to about 2 g).

Based on 1 lb of hydrophilic ointment base, optional ingredients in the composition may include Adijew™ NL-50 (50% aq., about 10 to about 100 cc, more preferably 25-35 cc), DL panthenol (about 1 to about 50 cc, more preferably about 5 to about 10 cc), glycerin USP (about 1 to about 20 cc, more preferably about 5 to about 10 cc), apricot kernel oil (about 0.1 to about 10 cc, more preferably about 1 to about 3 cc), vitamins A and D₃ (as a dispersion in corn oil, about 1×10⁶ IU/g to about 2×10⁷ IU/g of dispersion of vitamin A, more preferably about 6×10⁶ IU/g to about 1×10⁷ IU/g of dispersion of vitamin A; and 1×10⁵ IU/g to about 2×10⁶ IU/g of dispersion of vitamin D₃, more preferably about 6×10⁵ IU/g to about 1×10⁶ IU/g of dispersion of vitamin D₃, witch hazel extract (about 1 to about 50 cc, more preferably about 10 to about 20 cc), vitamin E acetate (1 g=1000 U, about 0.1 to about 20 cc, more preferably about 1 to about 4 cc), and ascorbyl palmitate (about 0.2 to about 10 g, more preferably, about 2 to about 4 g).

The above-mentioned composition is applied to the afflicted area of the skin in an amount of approximately at least 0.5 mL per 100 cm² per day, preferably at least about 1 mL per 100 cm² per day, and more preferably about 15 mL per 100 cm² per day. Preferably, the daily dosage does not exceed about 35 mL per 100 cm² per day.

When vitamin A is a component of the composition, it is administered in an amount of at least about 7000 IU/100 cm²/d, preferably at least about 14,000 IU/100 cm²/d, and more preferably about 200,000 IU/100 cm²/d. Preferably, the dosage, of vitamin A does not exceed about 500,000 IU/100 cm²/d.

When vitamin D₃ is a component of the composition, it is administered in an amount of at least about 500 IU/100 cm²/d, preferably at least about 1000 IU/100 cm²/d, and more preferably about 20,000 IU/100 cm²/d. Preferably, the dosage of vitamin D₃ does not exceed about 50,000 IU/100 cm²/d.

When vitamin E is a component of the composition, it is administered in an amount of at least about 2 IU/100 cm²/d, preferably at least about 5 IU/100 cm²/d, and more preferably about 75 IU/100 cm²/d. Preferably, the dosage of vitamin E does not exceed about 150 IU/100 cm²/d.

When ascorbyl palmitate is a component of the composition, it is administered in an amount of at least about 2.5 mg/100 cm²/d, preferably at least about 5 mg/100 cm²/d, and more preferably about 75 mg/100 cm²/d. Preferably, the dosage of ascorbyl palmitate does not exceed about 150 mg/100 cm²/d.

When quercetin dihydrate is a component of the composition, it is administered in an amount of at least about 2.5 mg/100 cm²/d, preferably at least about 5 mg/100 cm²/d, and more preferably about 75 mg/100 cm²/d. Preferably, the dosage of quercetin dihydrate does not exceed about 150 mg/100 cm²/d.

When DL-α-lipoic acid is a component of the composition, it is administered in an amount of at least about 1 mg/100 cm²/d, preferably at least about 2 mg/100 cm²/d, and more preferably about 35 mg/100 cm²/d. Preferably, the dosage of DL-α-lipoic acid does not exceed about 75 mg/100 cm²/d.

When green tea is a component of the composition, preferably it contains approximately 36% of catechins and polyphenols. The green tea is administered in an amount of at least about 0.5 mg/100 cm²/d, preferably at least about 1 mg/100 cm²/d, and more preferably about 20 mg/100 cm²/d. Preferably, the dosage of green tea does not exceed about 40 mg/100 cm²/d.

When rutin is a component of the composition, it is administered in an amount of at least about 0.5 mg/100 cm²/d, preferably at least about 1 mg/100 cm²/d, and more preferably about 20 mg/100 cm²/d. Preferably, the dosage of rutin does not exceed about 40 mg/100 cm²/d.

The total amount of flavonoid compounds administered in the composition is at least about 3 mg/100 cm²/d, preferably at least about 6 mg/100 cm²/d, and more preferably about 100 mg/100 cm²/d. Preferably, the dosage of all flavonoid compounds combined does not exceed about 200 mg/100 cm²/d.

The compositions of the present invention may also be formulated into a spray, mouth rinse, aerosol or inhalant. Such compositions may be prepared using well-known techniques. For these types of formulations, suitable carriers may include the following ingredients: saline with one or more preservatives, absorption promoters to enhance bioavailability, fluorocarbons and/or other conventional solubilizing or dispersion agents. These compositions are specialized forms of the topical compositions of the present invention and are designed for topical application to eczema or psoriasis including eczema or psoriasis that may occur in the mouth or nasal cavity.

The following examples are provided to describe the invention in further detail. These examples, which set forth a preferred mode presently contemplated for carrying out the invention, are intended to illustrate and not to limit the invention.

### EXAMPLE 1

A composition for use in the present invention is described in Table 1 below. These ingredients may be mixed with a suitable amount of a pharmaceutically acceptable carrier as described above.

TABLE 1

| Ingredient | Amount Employed |
|---|---|
| Hydrophilic Ointment Base | 1 lb |
| Ajidew NL-50 (50% aq.) | 25 cc |
| DL Panthenol | 5 cc |
| Glycerin USP | 5 cc |
| Apricot kernel oil | 3 cc |
| Vitamin A and D₃ dispersion in corn oil | 6 cc |
| Witch hazel extract | 12 cc |
| Vitamin E acetate (1 g = 1000 I.U.) | 2 cc |
| Ascorbyl Palmitate | 2 grams |
| Quercetin dihydrate | 2 grams |
| DL-α-Lipoic acid | 1 gram |
| Green Tea (36-50% C&P) | 500 mg |
| Rutin | 1 gram |

This composition can be administered 1-5 times daily for the reduction, treatment or at least partial prevention of reactive and inflammatory dermatoses such as eczema and psoriasis.

### EXAMPLE 2

A topical composition including a mixture of an hydrophilic ointment base, sodium acid phosphate moisturizing agent, a witch hazel extract carrier, glycerine, apricot kernel oil and DL-panthenol, as the pharmaceutically acceptable carrier and vitamins A and D₃, ascorbyl palmitate, α-lipoic acid and vitamin E acetate as the ingredients which have antioxidant properties and/or regulate cell differentiation and/or cell proliferation was prepared by cold compounding. The formulation of the topical composition is given in Table 2.

The topical composition was prepared by first placing the hydrophilic ointment base in a stainless steel bowl and mixing briskly until the ointment became creamy. Then, the sodium acid phosphate, panthenol, ascorbyl palmitate, glycerine, apricot kernel oil, vitamins A and D₃, quercetin, witch hazel extract, vitamin E acetate and α-lipoic acid were added, in that order. After each ingredient was added, mixing was continued until all traces of dry ingredients disappeared and a substantially homogeneous mixture was obtained. The final color was a consistent yellow and the cream had the consistency of cake frosting. The mixture was then placed in a sterile container. All containers and tools which contacted the topical composition or its ingredients were sterilized with, for example, a benzalkonium chloride, a sodium hypochlorite solution, or an iodine-based disinfectant.

This composition may be topically administered by applying a thin film of the composition to the areas of the skin afflicted with reactive and inflammatory dermatoses such as eczema and/or psoriasis. The topical composition may be applied three times daily, e.g., in the morning, at noon and before retiring. Patients applying the topical composition should experience less severe burning, irritation and redness in the areas of skin that were treated.

TABLE 2

| Ingredient | Amount Employed |
|---|---|
| Hydrophilic ointment base | 1 pound |
| 50% aqueous solution of Sodium acid phosphate | 25 cc |
| DL-panthenol | 5 cc |
| Glycerine | 5 cc |
| Apricot kernel oil | 3 cc |
| Witch hazel extract | 12 cc |
| α-Lipoic acid | 500 mg |
| Vitamin E acetate | 2 cc |
| Vitamin A and D₃ dispersion in corn oil | 6 cc |
| Ascorbyl Palmitate | 2 grams |
| Quercetin | 2 grams |

### EXAMPLE 3

Tables 3-8, below, set forth alternative topical formulations that may be employed in the method of the present invention. Certain of these formulations do not list every ingredient of the pharmaceutically acceptable topical carrier. These alternative formulations may be prepared using the procedure of Example 2.

TABLE 3

| Ingredient | Amount Employed |
|---|---|
| Ascorbyl Palmitate | 2 grams |
| Hesperidine | 2 grams |
| Rutin | 2 grams |
| Vitamin A and D₃ dispersion in corn oil | 3 cc |
| Vitamin E Acetate | 1 cc |
| DL Panthenol | 5 cc |

TABLE 4

| Ingredient | Amount Employed |
|---|---|
| Ascorbyl Palmitate | 2 grams |
| Ascorbyl Glucosamine | 1 gram |
| Luteolin | 4 grams |
| Vitamin A and D₃ dispersion in corn oil | 3 cc |
| Vitamin E acetate | 1 cc |
| DL Panthenol | 5 cc |

TABLE 5

| Ingredient | Amount Employed |
|---|---|
| Ascorbyl Glucosamine | 2 grams |
| Apigenin | 4 grams |
| Vitamin A and D₃ dispersion in corn oil | 3 cc |
| Vitamin E acetate | 1 cc |
| DL Panthenol | 5 cc |

TABLE 6

| Ingredient | Amount Employed |
|---|---|
| Ascorbyl Palmitate | 2 grams |
| γ-Linolenic acid | 500 mg |
| Rutin | 4 grams |
| Vitamin A and D₃ dispersion in corn oil | 3 cc |
| Vitamin E acetate | 1 cc |
| DL Panthenol | 5 cc |

TABLE 7

| Ingredient | Amount Employed |
|---|---|
| Ascorbyl Palmitate | 4 grams |
| Quercetin | 2 grams |
| Coenzyme Q10 | 500 mg |
| α-Lipoic acid | 50 mg |
| Vitamin A and D₃ dispersion in corn oil | 3 cc |
| Vitamin E acetate | 1 cc |
| DL Panthenol | 5 cc |

TABLE 8

| Ingredient | Amount Employed | |
|---|---|---|
| Vitamin A palmitate | 10,000 | IU as Vitamin A |
| Vitamin D₃ | 400 | IU |
| β-Carotene | 15,000 | IU |
| Vitamin E | 400 | IU |
| α-Lipoic acid | 150 | mg |
| Quercetin | 1200 | mg |
| Ascorbyl palmitate | 500 | mg |
| Curcumin | 15 | mg |
| Green tea (C&P) | 20 | mg |
| Chlorophyllin | 200 | mg |
| Carboxy ethyl sesquioxide of germanium | 100 | mg |
| Superoxide dismutase | 1,125 | mcg |

Certain patents and other publications have been cited in the present specification as background material. These patents and publications are incorporated herein by reference in their entirety. A standard reference text on pharmaceutical formulations, Remington's Pharmaceutical Sciences, 18^{th} Ed., Mack Publishing Co. 1990, is also incorporated herein by reference in its entirety.

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

What is claimed is:

1. A method for the reduction or treatment of reactive and inflammatory dermatoses comprising the step of administering, to a human afflicted by at least one dermatosis selected from the group consisting of reactive and inflammatory dermatoses, a composition which comprises a therapeutically effective amount of one or more flavonoids, and, optionally, a pharmaceutically acceptable carrier.

2. The method of claim 1, wherein the composition is administered topically.

3. A method as claimed in claim 2, wherein the flavonoids are selected from the group consisting of: 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; wogonin, and pharmaceutically acceptable salts thereof.

4. A method as claimed in claim 2, wherein the one or more flavonoids are selected from the group consisting of: quercetin, rutin, green tea, quercetrin, myricetin, kaempferol and myrecetrin.

5. The method of claim 2, wherein the one or more flavonoids are selected from the group consisting of quercetin, rutin, green tea, and any combination thereof.

6. The method of claim 5, wherein the one or more flavonoids comprise from 0.01% to 10% of the composition.

7. The method of claim 2, wherein the one or more flavonoids comprise quercetin, rutin, and green tea.

8. The method of claim 7, wherein the one or more flavonoids comprise from 0.01% to 10% of the composition.

9. A method as claimed in claim 2, wherein the composition further comprises one or more non-flavonoid antioxidants selected from the group consisting of: vitamin C and its esters; vitamin A and its esters; vitamin E and its esters; lipoic acid; carotenoids; chlorophyllin; coenzyme Q10; glutathione; and pharmaceutically acceptable salts thereof.

10. The method of claim 9, wherein the non-flavonoid antioxidant is lipoic acid.

11. The method of claim 9, wherein the non-flavonoid antioxidant is DL-α-lipoic acid.

12. The method of claim 11, wherein the one or more flavonoids are selected from the group consisting of quercetin, rutin, green tea, and any combination thereof.

13. The method of claim 12, wherein the one or more flavonoids comprise from 0.01% to 10% of the composition.

14. The method of claim 11, wherein the one or more flavonoids comprise quercetin, rutin, and green tea.

15. The method of claim 14, wherein the one or more flavonoids comprise from 0.01% to 10% of the composition.

16. The method of claim 2, wherein the composition further comprises an amount of one or more compounds effective to regulate at least one of cell differentiation and cell proliferation which is effective, when administered orally, to regulate at least one of cell differentiation and cell proliferation, said one or more compounds being selected from the group consisting of vitamin D₃, 1 (S),3(R)-dihydroxy-20(R)-(1-ethoxy-5-ethyl-5-hydroxy-2-heptyn-1-yl)-9, 10-seco-pregna-5(Z), 7(E), 10 (19)-triene, compounds that may be converted or metabolized into vitamin D₃ in the human body, metabolites thereof, and pharmaceutically acceptable salts thereof.

17. The method of claim 2, wherein the pharmaceutically acceptable carrier comprises a sufficient amount of at least one non-U.S.P. hydrophilic ointment base to form a substantially topical composition.

18. The method of claim 2, wherein the pharmaceutically acceptable carrier further comprises a sufficient amount of a panthenol selected from D-panthenol and DL-panthenol to promote penetration of one or more compounds of the composition into the skin.

19. The method of claim 2, wherein the pharmaceutically acceptable carrier comprises hydroxymethyl cellulose.

20. The method of claim 2, wherein the pharmaceutically acceptable carrier comprises an acrylic copolymer dissolved in polyethylene glycol.

21. The method of claim 2, wherein the composition comprises hydrophilic ointment base, sodium phosphoric acid, DL panthenol, glycerin, apricot kernel oil, vitamin A, vitamin D₃, witch hazel extract, vitamin E acetate, ascorbyl palmitate, quercetin dihydrate, DL-α-lipoic acid, green tea, and rutin.

22. The method of claim 2, wherein the composition comprises 1 lb of hydrophilic ointment base, 25 to 35 cc of 50% aqueous sodium phosphoric acid, 5 to 10 cc of DL panthenol, 5 to 10 cc of glycerin, 1 to 3 cc of apricot kernel oil, 6 to 10 cc of a dispersion of vitamin A and vitamin D₃ in corn oil, said dispersion including 1×10⁶ IU/g of vitamin A and 1×10⁵ IU/g of vitamin D₃, 10 to 20 cc of witch hazel extract, 1 to 4 cc of vitamin E acetate, wherein 1 g of vitamin E acetate provides 1000 IU of vitamin E, 2 to 4 g of ascorbyl palmitate, 2 to 4 g of quercetin dihydrate, 500 mg to 1 g of DL-α-lipoic acid, 500 mg to 1 g of green tea, and 1 to 2 g of rutin.

23. The method of claim 2, wherein the dermatosis is selected from the group consisting of eczema and psoriasis.

24. A method of facilitating the healing of wounds comprising administering topically, to a wound, an effective amount of a composition including a flavonoid and, optionally, a pharmaceutically acceptable carrier, to facilitate the healing of said wound.

25. A method of cleansing, beautifying, and improving the cosmetic appearance of the skin by topical administration to the skin of an effective amount of a composition including a flavonoid and, optionally, a pharmaceutically acceptable carrier, to improve the cosmetic appearance of the skin.
